(19) United States
(12) Patent Application Publication
(10) Pub. No.: US 2008/0249364 A1
(43) Pub. Date: Oct. 9, 2008

(54) ENDOSCOPIC INSTRUMENT

(75) Inventor: Eberhard KORNER, Knittlingen (DE)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: Richard Wolf GmbH, Knittlingen (DE)

(21) Appl. No.: 12/061,688

(22) Filed: Apr. 3, 2008

(30) Foreign Application Priority Data
Apr. 4, 2007 (EP) 07 007 041.2

(51) Int. Cl. A61B 1/008

(52) U.S. Cl. 600/141

(57) ABSTRACT

An endoscopic instrument having a flexibly designed tube shank is provided in which the tube shank is formed of several tube sections connected to one another in an articulated manner. Two adjacent tube sections are respectively connected to one another, in additional to the articulated connection (6), via at least one connection web (18), which is resilient in the axial direction of the tube shank.

### BACKGROUND OF THE INVENTION

The invention relates to an endoscopic instrument with a flexibly designed tube shank.

An endoscopic instrument which has a tube shank which may be bent or is flexible, is known, for example from German published patent application DE 195 34 112 A1. This tube shank consists of several tube sections which are engaged with one another in an articulated manner. The disadvantage with this instrument, however, is the fact that the individual tube sections need to be pre-tensioned in order to be able to transmit significant forces. With known instruments, a groove is provided in the wall of the tube shank for this, into which a pull element engages with a positive fit.

### BRIEF SUMMARY OF THE INVENTION

In view of the prior art, it is an object of the invention to provide an endoscopic instrument with a flexibly designed tube shank, wherein the tube shank has an as large as possible flexibility and is furthermore capable of transmitting larger forces.

The endoscopic instrument according to the invention comprises a flexibly designed tube shank, which is formed of several tube sections. The tube sections are arranged lying axially in succession and are respectively connected to one another in an articulated manner, so that the individual tube sections may be pivoted to one another in an articulated manner.

Additionally to the articulated connections, the individual tube sections are respectively connected to one another via at least one resilient connection web. The connection web or the connection webs respectively connect adjacent tube sections in a manner such that the connection web is designed in a resilient manner in the axial direction of the tube shank. The connection webs are connected to the adjacent tube sections, preferably in a physical manner, in particular with a material fit. The arrangement of the additional connection web has the advantage that a connection of the individual tube sections is created, which is firmer compared to a purely articulated connection, and permits a greater force transmission. Simultaneously, the resilient design of the connection web ensures an adequate movability between the individual tube sections, so that a large flexibility of the tube shank is maintained.

The connection webs ensure that, apart from the articulated connection, a firm connection between the individual tube sections exists, so that these are held together. Thus the connection web may, particularly preferably, transmit forces acting transversely to the axial direction, which may not be transmitted by the articulated connection.

Preferably, the connection webs are designed as one piece with the adjacent tube sections. This permits a firm connection of all tube sections, without complicated assembly procedures becoming necessary. Moreover, the attachment of the connection web to the tube sections may be designed in a particularly slim or thin manner, so that the free lumen inside of the tube shank may be designed as large as possible.

The connection webs are further preferably part of the peripheral walls of the tube sections. That is, the connection webs are themselves formed in the peripheral walls of the tube sections or tube shank. This design has the advantage that neither the outer diameter of the tube shank is increased in size, nor is the free lumen inside of the tube shank reduced in size, on account of the connection webs. The connection webs, seen in the axial direction, lie flush in the peripheral wall, on account of the design in the peripheral wall. In the radial direction, the connection webs preferably do not project to either the inside or to the outside, out of the peripheral wall.

Further preferably, the at least one connection web acts as a tension spring in the axial direction of the tube shank. Such a connection web is preferably arranged at the side of the tube shank, which is increased in length or extended on bending the tube shank. The result of this is that the connection web is extended on bending the tube shank, so that restoring forces are produced on account of the action as a tension spring, which automatically pull the tube shank back into its extended position or support the movement back into the extended position. A design as a compression spring is also possible, and then the connection web is arranged on the peripheral side toward which the deflection of the tube shank is effected, i.e., on the peripheral side which is compressed.

The deflection of the tube shank may be effected in a known manner by a pull, which extends on a peripheral side of the tube shank in the axial direction. This pull is preferably applied into a groove in the peripheral wall.

The connection webs between the several tube sections are particularly preferably situated all in the same peripheral region of the tube shank. That is, the individual connection webs respectively between adjacent tube sections, preferably in the axial direction, all lie on one line at the same angular position with respect to the longitudinal axis of the tube shank. Particularly preferably, it is here the case of the peripheral side or angular position, at which the tube shank is extended the most on bending, i.e., that angular region at which the individual tube sections are most remote from one another on bending.

The tube shank is preferably movable in a plane, wherein the lines of action of the resilient connection webs extend between the tube sections in this plane. That is, as previously described, the connection webs with their lines of action preferably extend along a line in the axial direction of the tube shank, wherein this one line is situated in the movement plane in which the tube shank is bent.

According to a further preferred embodiment, two respective tube sections which are adjacent to one another, are engaged with one another with a positive fit via hinge-like joints formed in the peripheral wall of the tube sections. These joints permit a large movability of the tube sections relative to one another in the bend direction or deflection direction of the tube shank. On account of the design of the joints within the peripheral wall, i.e., in a manner such that the joints do not project from the peripheral wall in the radial direction either to the outside or to the inside, the outer diameter of the instrument is thereby kept small, and simultaneously the free lumen inside is kept as large as possible. These hinge-like joints may be designed, for example, as is known from DE 195 34 112 A1.

The connection web between two adjacent tube sections preferably runs in a meandering manner with respect to its line of action. The spring effect in the direction of the line of action is achieved by this S-shaped or meandering design. Thereby, the spring effect is essentially not achieved by the extension of the material in the longitudinal direction, but chiefly by elastic bending in the S-design or meandering design of the connection web. The individual webs or limbs are thereby bent apart about their connection regions. With the design as a compression spring, the limbs are accordingly bent together, so that a compression of the connection web occurs. In this manner, a large length change, in particular lengthening or extension of the connection in the axial direction of the tube shank, is achieved when deflecting the tube shank. Moreover, a large stability in the lateral direction, i.e., the radial direction is ensured.

According to a further possible embodiment of the invention, two respective resilient connection webs are arranged in diametrically opposed peripheral regions, between two tube sections which are adjacent to one another, and these connection webs connect the tube sections to one another, wherein preferably one of the connection webs acts as a tension spring and the other as a compression spring in the direction of the longitudinal axis of the tube shank. Both connection webs are preferably arranged there such that the lines of action of their spring effect lie in the pivot plane or bending plane of the tube shank. There, the connection web acting as a tension spring is arranged on the side of the tube shank which is extended the most on bending or deflecting, while the connection web acting as a compression spring is arranged on the diametrically opposite side of the tube shank, toward which the tube shank is deflected. This is the side which is compressed the most on deflecting. Thus, the connection web acting as a compression spring is compressed on deflection, while the connection web acting as a tension spring is extended. In this manner, a larger restoring force is produced, which moves the tube sections back again into their extended initial position, or supports such a restoring movement. Moreover, a large stability between the two tube sections, in particular in the radial direction, is achieved by the arrangement of two connection webs, so that as a whole, a larger force transmission is possible with the tube shank.

According to a further preferred embodiment, the connection webs have different spring characteristics between different pairs of tube sections which are adjacent to one another. Seen over the length of the tube shank, several or a multitude of tube sections are arranged lying in succession, which are connected respectively to one another via at least one connection web. According to this preferred embodiment, these connection webs are not all the same, and in particular they are not designed with the same spring characteristics. Thus, one may create a tube shank which has different degrees of stiffness or flexibility characteristics over its length. Regions may be provided, which are designed stiffer than other regions. Moreover, one may thus create regions in which these are bent to a greater extent than in other regions, when deflecting or bending the tube shank. Thus, the instrument, with regard to its flexibility characteristics, may be optimally adapted to the desired application purpose.

Particularly preferably, the connection webs in the peripheral walls are formed by introducing separating gaps into the peripheral walls of the tube sections. Thus, it is possible to design the connection webs as one piece with the tube sections, by incorporating suitable incisions into the wall of the tube, from which the tube sections are formed, the incisions shaping the connection webs. Thus, the connection webs are formed directly in the peripheral wall. A very flexible shaping for the connection webs is possible, depending on the shape of the incisions. The joints between the individual tube sections, and the separating gaps situated between the tube sections, are preferably cut into a continuous tube. Thus, all tube sections, the joints connecting these as well as connection webs, may be cut directly into a single-piece, continuous tube. This tube may, moreover, extend without incisions as one piece further proximally, so that a continuous tube shank of the instrument may be created, which is flexible or deflectable at the distal end, and is designed in a rigid manner at the proximal end. The separating gaps or the incisions may, for example, be formed by laser beam cutting or electrode beam cutting.

Further preferably, separating gaps are formed between the individual tube sections and respectively, in one separating gap, preferably in all separating gaps in the same peripheral region of the tube shank, have a greater width in the direction of the longitudinal axis of the tube shank than in the other peripheral regions and in particular than in a diametrically opposite peripheral region. That is, the separating gaps are designed wider in the region toward which the tube shank is to be deflected or bent, so that there, the individual tube shanks are spaced further from one another in the extended condition of the tube shank. In the bent condition of the tube shank, the individual tube segments in this region are moved toward one another, so that the separating gaps in the completely bent condition are preferably almost completely closed in this region, i.e., the individual tube sections bear on one another. Simultaneously, on the opposite peripheral side, the tube sections move apart, so that here, the separating gaps are widened on bending. That is, the individual tube sections which bear almost completely on one another in the extended position, distance themselves from one another in this peripheral region of the tube shank, in which the largest extension occurs. Preferably, the elastic or resilient connection web, which is extended on bending, is also arranged in this region.

The widening separating gaps are preferably designed such that the separating gaps, proceeding from the two joint points arranged diametrically opposite one another, widen to the peripheral region which lies exactly between these joint points. The separating gap on the opposite peripheral side is only designed in a very narrow manner, so that here the tube sections essentially bear on one another in the stretched condition of the tube shank.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a lateral view of a tube shank for an endoscopic instrument according to a first embodiment of the invention,

FIG. 2 is a simplified lateral view of the tube shank according to FIG. 1,

FIG. 3 is an enlarged detail view of the cutout III in FIG. 2,

FIG. 4 is a sectioned view along line IV-IV in FIG. 3,

FIG. 5 is a bent representation of a tube shank for an endoscopic instrument, according to a second embodiment of the invention,

FIG. 6 is an enlarged representation of the articulated connection of two tube shanks, according to the embodiment in FIG. 5,

FIG. 7 is a lateral view of the tube shank according to FIG. 5, seen from the side of the connection webs,

FIG. 8 is a lateral view of the tube section according to FIG. 7, rotated by 90°,

FIG. 9 is a sectioned view of the tube shank according to FIG. 8,

FIG. 10 is a view of the tube shank according to FIGS. 5 to 9, in the bent condition,

FIG. 11 is a sectioned view of the bent tube shank according to FIG. 10,

FIG. 12 is a lateral view of a tube shank for an endoscopic instrument according to a third embodiment of the invention,

FIG. 13 is a lateral view of the tube shank according to FIG. 12, rotated by 90°,

FIG. 14 is a lateral view of the tube shank according to FIGS. 12 and 13, once again rotated by 90° with respect to FIG. 13, and

FIG. 15 is a tube shank according to a further embodiment of the invention.

### DETAILED DESCRIPTION OF THE INVENTION

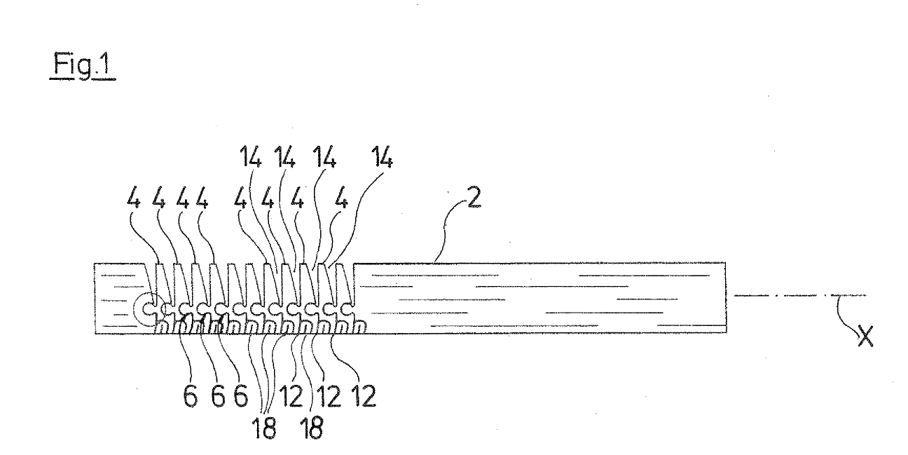

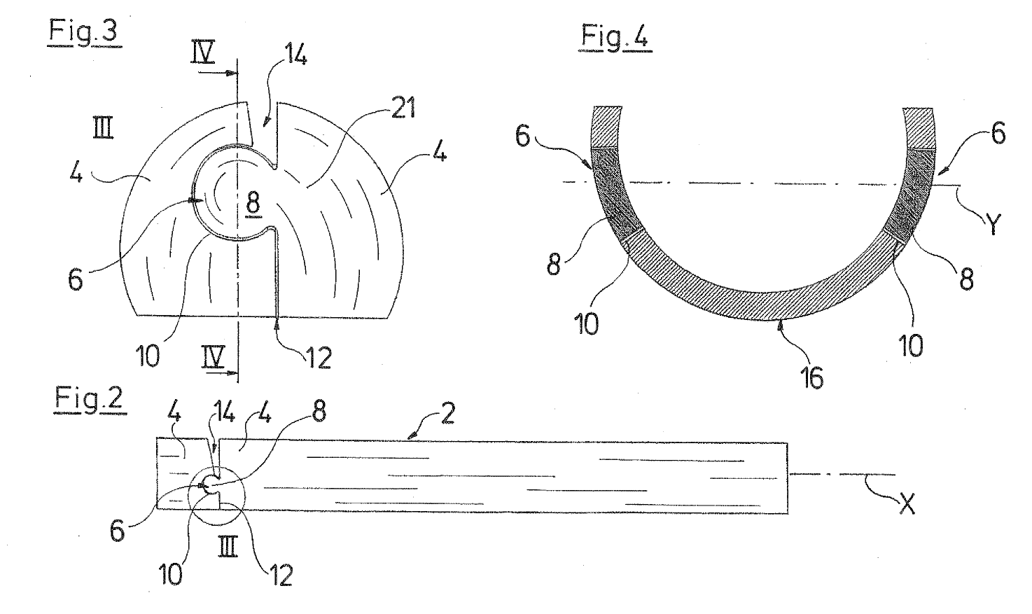

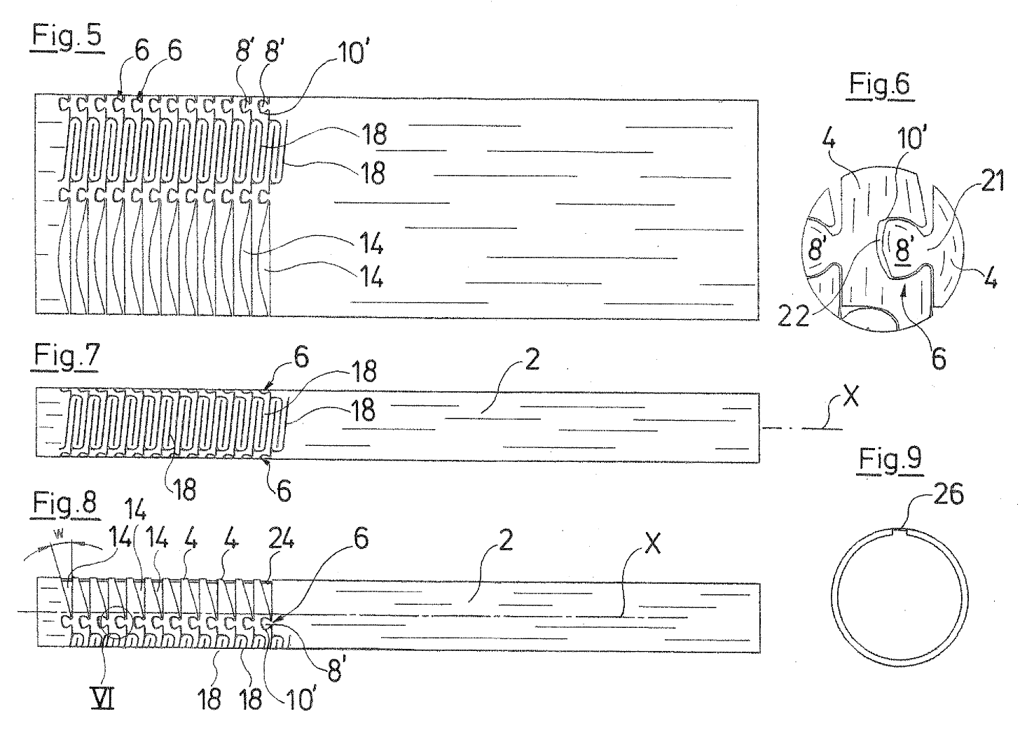

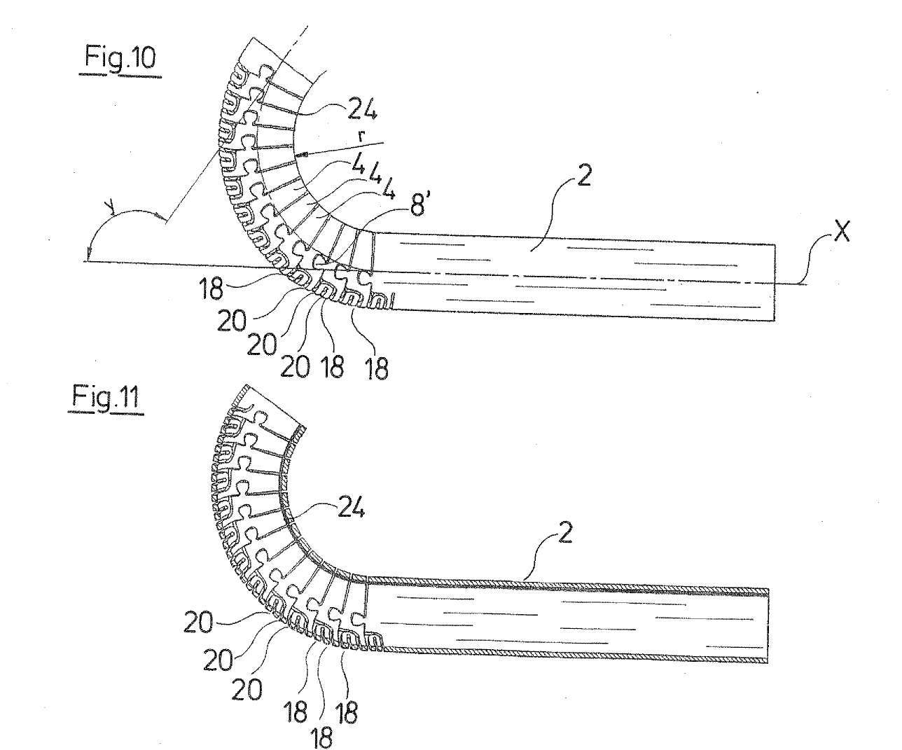

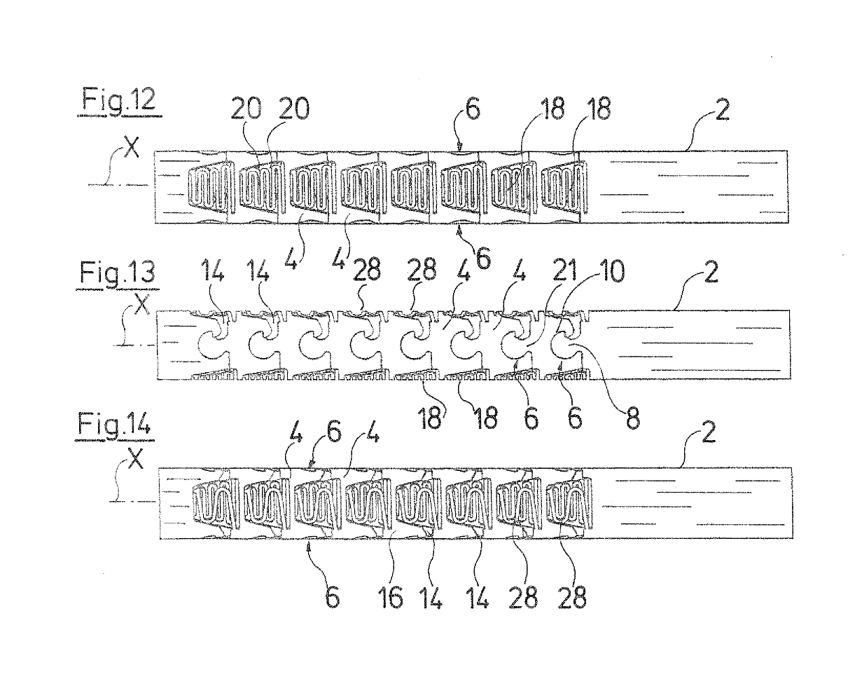

The basic construction of an endoscopic instrument or a tube shank for such an instrument according to a first preferred embodiment of the invention is explained by the FIGS. 1 to 4. One should take note that in each case, only the tube shank of the instrument is schematically represented in FIGS. 1 and 2, wherein only a few of the multitude of tube sections 4 of the tube shank 2, from which the flexible part of the tube shank 2 is formed, are represented in FIG. 1. It is to be understood here that still further tube sections of a corresponding design may be formed in the tube shank 2. Accordingly, only a single connection between the tube sections 4 is represented in FIG. 2, for illustrating the design of this connection.

The flexible or articulated part of the tube shank 2 is formed of a plurality of tube sections 4 lying in succession in the axial (longitudinal) direction X. The tube sections 4 are respectively engaged with one another with a positive fit at joints 6 formed diametrically opposite one another in the peripheral wall. This articulated connection is constructed similarly as is known, for example, from DE 195 34 112 A1 or from DE 195 35 179 A1.

As shown more clearly in FIGS. 2 and 3, the joint connections 6 in each case have a circular pivot 8 on a tube section, and a corresponding circular recess 10 on an adjacent tube section 4. The pivot 8 engages into the recess 10 in a fitting manner and with a positive fit, so that it may rotate in the recess 10. For this, the separating gap 12 between the two tube sections 4 is designed such that wider notches 14 are formed on one side of the periphery, proceeding from the two diametrically opposite joint connections 6. That is, the adjacent tube sections 4 are spaced further from one another. Thereby, the notches 14 are formed such that they widen proceeding from the two joint connections 6, so that the largest width in the longitudinal direction X is situated essentially at an angular position offset by 90° to the two joint connections 6 on the periphery of the tube shank 2. The notches 14 permit two respectively adjacent tube sections 4 to be pivoted about the joint connections 6 toward the peripheral side on which the notches 14 are formed. The individual tube sections 4 respectively pivot to one another in an articulated manner about a pivot axis which extends through the middle point of the pivot 8 transversely or normally to the longitudinal axis X. As a whole, a flexible deflection of the tube shank is achieved, as shown in FIGS. 10 and 11, by pivoting the individual tube sections 4 to one another. Thereby, the tube shank curves about an external, fictive point with a radius r, wherein the radius r reduces in size with an increasing curvature. The maximal curvature with the maximal pivot angle γ of each tube shank 2 is achieved when the end-side edges of adjacent tube sections 4, which lie opposite one another, come to bear on one another, in the region of the notches 14. The separating gap 12 on the diametrically opposite side of the notch 14 simultaneously widens.

As may be recognized in the sectioned view in FIG. 4, the joint connections 6 are formed completely in the wall 16 of the tube sections 4 or of the tube shank 2. In this manner, the cross-section which is available is utilized in an optimal inner. This means that on the one hand the outer diameter of the instrument is not increased in size by the joint connections, and on the other hand the free lumen inside of the tube shank 2 is not reduced in size by the joint connections 6. With the embodiment shown in FIGS. 1 to 4, the pivots 8 and the recesses 10 are curved with the course of the peripheral wall 16. This has the effect that the pivots 8 and the recesses 10 on their radially inner lying side in the extension direction of the peripheral direction 16 have a smaller diameter than on the outer side. That is, the pivots 8 as a whole are thus designed conically toward the inside of the tube shank 2. In this manner, a lateral displacement in the diameter direction or the direction of the pivot axis Y transversely or normally to the longitudinal axis X is prevented.

According to the invention, the individual tube sections 4 are not completely separated from one another by the separating gaps 12. Rather, the separating gaps 12, at the side which is diametrically opposite the notches 14, are formed such that respectively continuous connection webs 18 between the individual tube sections 4 are formed, which connect adjacent tube sections 4 to one another. That is, the tube sections 4 are not completely separated from one another, but additionally to the joint connections 6, are held together by the connection webs 18. The connection webs 18 extend in a zigzag manner or in an S-shaped or meandering manner with respect to the longitudinal axis X between adjacent tube sections 4. An elasticity or spring effect of the connection webs 18 in the direction of the longitudinal axis X is achieved in this manner.

Thus, the connection webs 18 may extend on deflecting or pivoting the flexible section of the tube section 2, with which the individual tube sections 4 pivot about their pivots 8, as described above. The tube sections 4 on pivoting, distance themselves from one another in the region between the tube sections 4, in which the connection webs 18 are arranged, while they approach one another in the region of the notch 14. The extension of the connection webs 18, which is thereby effected by this, is effected by the fact that a bending is effected in the region of the curves or radii of the connection webs 18 in the S-shaped course of the connection webs 18, wherein the straight sections or limbs between these radii of the connection webs 18 distance themselves further from one another. This elastic deformation of the connection webs 18 produces a restoring force or spring force, which ensures a restoring of the tube shank 2 into the extended position shown in FIG. 1, as soon as the force for deflecting is released, or at least simplifies this restoring movement. The extended condition of the connection webs 18 is to be recognized in FIGS. 10 and 11, in which the tube shank 2 is deflected or curved. There, one may recognize that gaps 20 form by the extension of the connection webs 18 between the limbs of the connection webs 18, which run in the peripheral direction, while these parts of the connection webs 18 with the exception of the narrow separating gaps 12, essentially directly bear on one another in the extended condition which is shown in the FIGS. 1, 5, 7 and 8.

A second embodiment of the invention is shown in FIGS. 5 to 8 as well as 10 and 11, which differs from the embodiments explained by FIGS. 1 to 4 only by the design of the joint connections 6. All other details, and in particular the design of the connection webs 18, are identical. With the design according to FIGS. 5 to 8 and 10 to 11, the pivots 8′ are not circular and the recesses 10′ accordingly are likewise not designed in a circular manner. The pivots 8′ have an arc-like outer contour at that side at which they are connected to an adjacent tube section 4 via a web 21. The pivot 8′ is designed shortened or cut-off in the region which is distant to the web 21. Accordingly, the recess 10′ only has an arc-like contour in the region adjacent to its opening, and the base of the recess 10′ is likewise cut-off, wherein a free space 22 remains in this region between the base of the recess 10′ and the end-side of the pivot 8′, as is clearly seen in the enlargement of the cutout in FIG. 6. This free space 22 permits the movement of the pivot 8′ in the recess 10′, wherein the adjacent tube sections 4, as explained by FIGS. 1 to 4, pivot to one another, which is shown in FIGS. 10 and 11. Here too, the pivots 8′ and the recesses 10′ are designed as shown in FIG. 6. The pivoting, however, would function in the same manner as with the pivots 8 shown in FIGS. 1 to 4.

The second embodiment of the invention shown in FIGS. 5 to 8 as well as 10 and 11, on account of the joint connections which are not completely designed in a circular manner, permits narrower tube sections 4 in the longitudinal direction X, whereby, as a whole, smaller deflection radii may be achieved.

The meandering course of the connection webs 18 is seen more clearly in FIGS. 5 and 7. There, FIG. 5 shows a view in which the peripheral wall of the tube section 2 is cut-open and unwound.

The deflection or curvature of the tube shank 2 is effected by a pull element 24, which extends in the longitudinal direction X in a groove 26 in the peripheral wall 16 of the tube shank 2 and tube sections 4. This pull element 24 is fastened at the distal end of the tube shank and, furthermore, is movable relative to the tube shank. If the pull element 24 is pulled proximally, in this manner the tube shank in its movable region which is formed by the tube sections 4, is deflected as shown in FIGS. 10 and 11. This pull element is not represented In FIGS. 1, 2 and 9 as well as FIGS. 13 and 14, but it is to be understood that it is arranged there in a corresponding manner. The restoring of the tube shank 2 into its extended position is effected first by releasing the force acting on the pull element 24 in the proximal direction. On account of its elasticity, the connection webs 18 seek to move back into their initial position shown in FIGS. 1, 5 to 8, as well as 12 to 14, i.e., the connection webs 18 here produce a restoring force or a part of the restoring force. Additionally, the pull element 24 may be designed as a pull element/push element, so that it may also transmit a push force in the distal direction, by which the tube shank 2 may be moved back into its extended position with a pivot angle γ=0°.

The connection webs 18 between the individual tube sections 4, given a constant flexibility or movability of the tube shank 2, ensure an increased stability, which permits a larger force transmission, since they permit a larger force transmission between the tube sections 4 which are movable relative to one another, in particular in the lateral, i.e., radial or peripheral direction with respect to the longitudinal axis X.

FIGS. 12 to 14 show a further embodiment of the invention in which, on the one hand, the connection webs 18 are designed somewhat differently than with the previously described embodiments. The connection webs 18 comprise more windings, and the windings are spaced slightly from one another, also in the extended initial position of the tube shank 2 shown in FIGS. 12 to 14, wherein the gaps 20 between the individual windings or limbs of the connection webs 18 extending in the peripheral direction, distance further from one another on curvature, similarly to FIGS. 10 and 11. Furthermore, the pivots 8 and the associated recesses 10, although likewise being designed of a circular contour, however have a hook-like shape close to the web 21, in contrast to the embodiment in FIGS. 1 to 4. The pivoting is, however, effected just as is described by FIGS. 1 to 4. The main difference to the previously described embodiments lies in the fact that further connection webs 28 are formed between adjacent tube sections 4, diametrically opposite the connection webs 18 in the wall 16 of the tube sections 4.

The connection webs 28 are designed as the connection webs 18 in the peripheral wall 16 of the tube shank 2 itself, and likewise have an S-shaped or meandering course with respect to the longitudinal axis X. However, with the connection webs 28, the windings are larger and are spaced further from one another in the shown rest position, since the connection webs 18 act as a compression spring in the longitudinal direction X. On pivoting, similarly to FIGS. 10 and 11, the tube sections 4 are pivoted about the joint connections 6, so that they approach one another in the regions of the notches 14. Since the connection webs 28 are arranged on the peripheral side at which the recesses 14 also have their greatest width, the connection webs 18 are thereby compressed, i.e., their windings approach one another. This is effected by a bending in the region of the radii of curvature between the individual limbs of the connection webs 28. Simultaneously with the extension of the connection webs 18, the connection webs 28 thus produce a further restoring force by the fact that, on account of their elasticity, they strive to press the tube sections 4 in the region of the notches 14 apart again. Moreover, the connection webs 28, which connect the adjacent tube sections 4 to one another as one piece according to the arrangement of the connection webs 18, ensure an additional stability in the diametrical direction or peripheral direction.

In the shown examples, a deflection or curvature of the tube shank 2 is only provided in one plane and in one direction. For this reason, the joint connections 6 in the shown examples are all arranged lying in the same peripheral region in the longitudinal direction X on one line. Accordingly, the connection webs 18 and 28 are also arranged lying in the longitudinal direction on one line. However, it is conceivable to permit a deflection in several directions, wherein then individual joint connections 6 and, thus accordingly, the notches 14 as well as the connection webs 18 and 28, may be arranged offset in the peripheral direction.

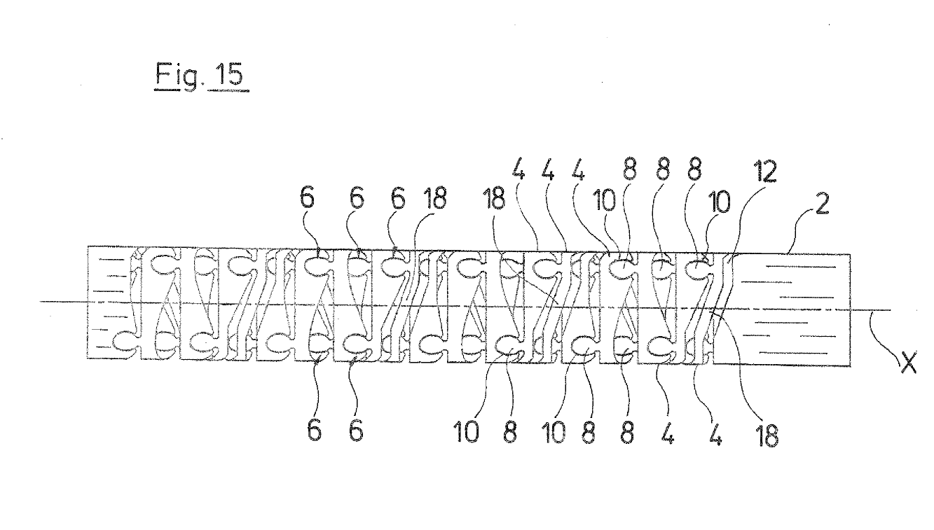

One example of such an embodiment is shown in FIG. 15. There, the tube sections 4 are connected to one another such that the joint connections 6, consisting of pivot 8 and recess 10, are arranged between adjacent tube sections 4 alternately offset to one another by 90° with respect to the longitudinal axis. A pivoting in two planes directed normal to one another is possible in this manner. There, the pivot axes directed normally to one another always alternate. This means that, seen in the direction of the longitudinal axis, a first pivot axis between a first and a second tube section is directed in a first direction, while the next pivot axis between the second and a third tube section is directed rotated or offset by 90° with respect to the longitudinal axis, and so on. In this manner, a deflection of the distal end of the tube shank is possible in any direction. Instead of arranging pivot axes alternately offset by 90° to one another, it is also possible to form several groups of tube sections, wherein the individual groups respectively consist of several tube sections, between which the pivot axes are directed in the same direction. The individual groups then differ by the fact that the pivot axes of the different groups are directed at an angle to one another, preferably at an angle of 90° to one another.

Moreover, it is also possible to arranged a further recess 14 diametrically opposite in the region of the connection webs 18, so that a pivoting is also possible in the opposite direction. Then the connection web 18 would act as a compression spring corresponding to the connection web 28.

Moreover, in the shown examples, all tube sections 4 and in particular all connection webs 18 and 28 are designed in an identical manner over the length of the tube shank 2. However, it is conceivable to design individual connection webs 18 and 28 differently over the length of the tube shank 2, in order to provide different degrees of stiffness, spring forces and/or joint characteristics between the individual tube sections 4. Thus, the instrument may be optimally adapted to the desired application purpose by the design of its tube shank 2. It is also to be understood that the joint connections 6 may also be designed in a different manner, in particular shaped in a different manner.

The tube shank 2 with the notches 14, with the joint connections 6 and with the connection webs 18 and 28, are manufactured from a tube by cutting separating gaps 12, as well as recesses and gaps which form the connection webs 18, into the tube. This may be effected, for example, by laser beam cutting or electron beam cutting. It is simply possible to introduce complex shaping into the wall 16 of the tube shank 2 and to design the individual tube sections 4 as one piece and simultaneously to ensure the movement ability about the joint connections in this manner.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I/we claim:

1. An endoscopic instrument having a flexibly designed tube shank (2), wherein the tube shank (2) comprises several tube sections (4) respectively connected to one another by an articulated connection (6), and
wherein two adjacent tube sections (4), are respectively connected to one another, in addition to the articulated connection (6), via at least one connection web (18) which is resilient in an axial direction (X) of the tube shank (2).

2. The endoscopic instrument according to claim 1, wherein the connection web (18) comprises one piece with the adjacent tube sections (4).

3. The endoscopic instrument according to claim 1, wherein the connection web (18) is part of a peripheral wall (16) of the tube sections (4).

4. The endoscopic instrument according to claim 1, wherein the connection web (18) acts as a tension spring in the axial direction (X) of the tube shank (2).

5. The endoscopic instrument according to claim 1, wherein the connection webs (18) between several of the tube sections (4) all lie in a same peripheral region of the tube shank (2).

6. The endoscopic instrument according to claim 1, wherein the tube shank (2) is movable in a plane, wherein lines of action of the connection webs (18) extend between the tube sections (4) in this plane.

7. The endoscopic instrument according to claim 1, wherein two adjacent tube sections (4) are respectively engaged with one another with a positive fit, via articulated connections (6) in a form of hinge-like joints formed in a peripheral wall (16) of the tube sections (4).

8. The endoscopic instrument according to claim 1, wherein the connection web (18) runs in a meandering manner with respect to its line of action.

9. The endoscopic instrument according to claim 1, wherein two adjacent tube sections (4) are connected to one another respectively via two resilient connection webs (18, 28) arranged in diametrically opposite peripheral regions, wherein one connection web acts as a tension spring (18) and another connection web acts as a compression spring (28), in the axial direction (X) of the tube shank (2).

10. The endoscopic instrument according to claim 1, wherein the connection webs (18, 28) between different pairs of tube sections (4) adjacent to one another have different spring characteristics.

11. The endoscopic instrument according to claim 1, wherein the connection webs (18, 28) in peripheral walls (16) of the tube sections (4) are formed by introducing separating gaps into the peripheral walls.

12. The endoscopic instrument according to claim 1, wherein separating gaps (12) are formed between individual tube sections (4), and wherein separating gaps (12) in a same peripheral region (14) of the tube shank (2) have a width which is wider in the axial direction (X) of the tube shank (2) than in another peripheral region.

13. The endoscopic instrument according to claim 12, wherein the other peripheral region is a diametrically opposite peripheral region.
